SEQUENCE LISTING (1 sequence)

SEQ ID NO: 1 - DNA, 2203 bp, Homo sapiens
gtctgcatccggtggccacagagcaacttcctctagagggagctgattggagccgggtgccgctggcacctctatgatcactggagtctcgcgggtccctcgggctgcacagggacaagtaaaggctacatccagatgccgggaatgcactgacgcccattcctggaaactgggctcccactcagcccctgggagcagcagccgccagcccctcgggacctccatctccaccctgctgagccacccgggttgggccaggatcccggcaggctgatcccgtcctccactgagacctgaaaaatggcttcggggcaaggcccaggtcctcccaggcaggagtgcggagagcctgccctgccctctgcttctgaggagcaggtagcccaggacacagaggaggttttccgcagctacgttttttaccgccatcagcaggaacaggaggctgaaggggtggctgcccctgccgacccagagatggtcaccttacctctgcaacctagcagcaccatggggcaggtgggacggcagctcgccatcatcggggacgacatcaaccgacgctatgactcagagttccagaccatgttgcagcacctgcagcccacggcagagaatgcctatgagtacttcaccaagattgccaccagcctgtttgagagtggcatcaattggggccgtgtggtggctcttctgggcttcggctaccgtctggccctacacgtctaccagcatggcctgactggcttcctaggccaggtgacccgcttcgtggtcgacttcatgctgcatcactgcattgcccggtggattgcacagaggggtggctgggtggcagccctgaacttgggcaatggtcccatcctgaacgtgctggtggttctgggtgtggttctgttgggccagtttgtggtacgaagattcttcaaatcatgactcccaagggtgccctttggggtcccggttcagacccctgcctggacttaagcgaagtctttgccttctctgttcccttgcaggggtcccccctcaagagtacagaagctttagcaagtgtgcactccagcttcggagggcccctgcgtgggggccagtcaggctgcagaggcacctcaacattgcatggtgctagtgggccctctctctgggcccaggggctgtggccgtctcctccctcagctctctgggacctccttagccctgtctgctaggcgctggggagactgataacttggggaggcaagagactgggagccacttctccccagaaagtgtttaacggttttagctttttataatacccttgtgagagcccattcccaccattctacctgaggccaggacgtctggggtgtggggattggtgggtctatgttccccaggattcagctattctggaagatcagcaccctaagagatgggactaggacctgagcctggtcctggccgtccctaagcatgtgtcccaggagcaggacctactaggagaggggggccaaggtcctgctcaactctacccctgctcccattcctccctccggccatactgcctttgcagttggactctcagggattctgggcttggggtgtggggtggggtggagtcgcagaccagagctgtctgaactcacgtgtcagaagcctccaagcctgcctcccaaggtcctctcagttctctcccttcctctctccttatagacacttgctcccaacccattcactacaggtgaaggctctcacccccatccctgggggccttgggtgagtggcctgctaaggctcctccttgcccagactacagggcttaggacttggtttgttatatcagggaaaaggagtagggagttcatctggagggttctaagtgggagaaggactatcaacaccactaggaatcccagaggtgggatcctccctcatggctctggcacagtgtaatccaggggtgtagatgggggaactgtgaatacttgaactctgttcccccaccctccatgctcctcacctgtctaggtctcctcagggtggggggtgacagtgccttctctattgggcacagcctagggtcttgggggtcaggggggagaagttcttgattcagccaaatgcagggaggggaggcagatggagcccataggccaccccctatcctctgagtgtttggaaataaactgtgcaatcccctcaccctgaaaaaaaaaaa

(19) United States
(12) Patent Application Publication
(10) Pub. No.: US 2010/0247530 A1
(43) Pub. Date: Sep. 30, 2010

(54) COMPOSITIONS AND METHODS FOR PREVENTING AND TREATING PRESBYCUSIS

(75) Inventors: Shinichi Someya, Madison, WI (US); Tomas A. Prolla, Madison, WI (US)

Correspondence Address: Casimir Jones, S.C., 2275 DEMING WAY, SUITE 310, MIDDLETON, WI, 53562, US

(73) Assignee: MARINE BIO CO., LTD., Tokyo (JP)

(21) Appl. No.: 12/727,902

(22) Filed: Mar. 19, 2010

Related U.S. Application Data
(60) Provisional application No. 61/161,836, filed on Mar. 20, 2009.

(51) Int. Cl. A61K 39/395; C07H 21/02; C12N 15/11; A61P 25/00; A61P 27/16

(52) U.S. Cl. 424/133.1; 514/44 A; 514/44 R; 424/172.1; 424/152.1; 424/178.1; 424/183.1

(57) ABSTRACT

The present invention provides compositions and methods for preventing and treating presbycusis. In particular, the present invention provides compositions and methods for preventing and treating presbycusis by inhibiting Bak.

### CROSS REFERENCE TO RELATED APPLICATIONS

This application claims priority to U.S. Provisional Patent Application Ser. No. 61/161,836, filed Mar. 20, 2009, which is herein incorporated by reference in its entirety.

### FIELD OF THE INVENTION

The present invention provides compositions and methods for preventing and treating presbycusis. In particular, the present invention provides compositions and methods for preventing and treating presbycusis by inhibiting Bak.

### BACKGROUND OF THE INVENTION

Age-related hearing loss, also known as presbycusis, is a common feature of mammalian aging and is the most frequently occurring sensory disorder in the elderly population (Gates & Mills, Lancet 366, 1111 (2005), herein incorporated by reference in its entirety). Presbycusis is characterized by age-dependent decline of auditory function and the losses of hair, spiral ganglion (SG) neuronal, and stria vascularis cells in the cochlea of the inner ear (Gates & Mills, Lancet 366, 1111 (2005), Yamasoba et al., Hear Res 226, 185 (2007), herein incorporated by reference in their entireties). It is estimated that more than 40% of individuals over 65 years of age have presbycusis (Gates & Mills, Lancet 366, 1111 (2005).) and the number of people suffering from this disorder is rapidly growing as numbers of older individuals in industrialized nations increases.

The oxidative stress theory of aging is one of the most widely acknowledged theories of aging and postulates that aging is the result of accumulated oxidative damage caused by oxidants or reactive oxygen species (ROS) (Beckman & Ames, Physiol Rev 78, 547 (1998), Harper et al., Acta Physiol Scand 182, 321 (2004), Finkel & Holbrook, Nature 408, 239 (2000), herein incorporated by reference in their entireties). The importance of this theory is supported by observations that enhancing antioxidant defense can increase longevity in several organisms (Finkel & Holbrook, Nature 408, 239 (2000), Lin & Beal, Nature 443, 787 (2006), herein incorporated by reference in their entireties). In Drosophila, overexpression of the mitochondrial antioxidant enzyme manganese superoxide dismutase (MnSOD) extends lifespan (Lin & Beal, Nature 443, 787 (2006).), and overexpression of glutathione reductase, an enzyme involved in the generation of cellular antioxidant reduced glutathione, increases resistance to oxidative stress and extends lifespan under hyperoxic conditions (Mockett et al., Faseb J 13, 1733 (1999), herein incorporated by reference in its entirety). In mice, overexpression of the catalase enzyme experimentally targeted to mitochondria extended median and maximum lifespan by 20% (Schriner et al., Science 308, 1909 (2005), herein incorporated by reference in its entirety). It is widely accepted that mitochondria are a major source of ROS and a major site of ROS-induced oxidative damage (Harper et al., Acta Physiol Scand 182, 321 (2004), Lin & Beal, Nature 443, 787 (2006).). Indeed, an age-associated increase in oxidative damage to mitochondrial DNA (mtDNA) has been well documented (Beckman & Ames, Physiol Rev 78, 547 (1998), Lin & Beal, Nature 443, 787 (2006).). The accumulation of mtDNA damage can result in dysfunctional mitochondrial respiratory chain complexes which lead to increased ROS generation and further ROS-induced oxidative damage, a cycle which eventually leads to programmed cell death or apoptosis (Lin & Beal, Nature 443, 787 (2006).).

The process of apoptosis is postulated to play a key role in the declining physiological function of multiple organs with aging (Kujoth et al., Science 309, 481 (2005), Dirks & Leeuwenburgh, Free Radic Biol Med 36, 27 (2004), herein incorporated by reference in their entireties), and levels of apoptosis increase during aging in mouse cochlea (Someya et al., Neurobiol Aging 29, 1080 (2008), herein incorporated by reference in its entirety). However, despite the growing evidence linking oxidative stress, apoptosis, and aging, specific mechanisms are unknown and direct evidence demonstrating a causal role is lacking.

Apoptosis can occur through two major pathways: the intrinsic pathway, also known as the mitochondrial pathway, which is initiated when the outer mitochondrial membrane (OMM) loses its integrity, or the extrinsic pathway which is initiated through ligand binding to cell surface receptors (Youle & Strasser, Nat Rev Mol Cell Biol 9, 47 (2008), herein incorporated by reference in its entirety). In mammals, mitochondria play a major role in apoptosis that is regulated by Bcl-2 family members (Youle & Strasser, Nat Rev Mol Cell Biol 9, 47 (2008).). Of the Bcl-2 family members, the proapoptotic proteins Bak and Bax are postulated to play a central role in promoting mitochondrial-mediated apoptosis (Youle & Strasser, Nat Rev Mol Cell Biol 9, 47 (2008), Lindsten et al., Mol Cell 6, 1389 (2000), herein incorporated by reference in their entireties). These Bcl-2 proteins promote permeabilization of the OMM, leading to cytochrome c release in the cytosol (Youle & Strasser, Nat Rev Mol Cell Biol 9, 47 (2008)).

### SUMMARY

The present invention provides compositions and methods for preventing and treating presbycusis. In particular, the present invention provides compositions and methods for preventing and treating presbycusis by inhibiting Bak.

In some embodiments, the present invention provides a method of providing medical treatment to a subject comprising inhibiting Bak. In some embodiments, providing medical treatment comprises preventing presbycusis. In some embodiments, providing medical treatment comprises treating presbycusis. In some embodiments, inhibiting Bak comprises inhibiting expression of the Bak gene. In some embodiments, inhibiting Bak comprises inhibiting the activity of the Bak protein. In some embodiments, inhibiting Bak comprises administering Bak-inhibiting compounds to a subject. In some embodiments, inhibiting Bak comprises administering siRNA to a subject. In some embodiments, inhibiting Bak comprises introducing a gene to a subject which inhibits the expression of the Bak gene in the subject. In some embodiments, a gene introduced to a subject inhibits the expression of the Bak gene in the cochlear cells the subject. In some embodiments, inhibiting Bak in a subject inhibits one or more neurodegenerative diseases. In some embodiments, the present invention provides a method of inhibiting presbycusis in a mouse, companion animal (e.g., dog or cat) or other mammal. In some embodiments, the present invention provides a method of treating or preventing presbycusis in a human. In some embodiments, inhibiting Bak in a subject comprises inhibiting Bak in cochlear cells. In some embodiments, bak in prevents or treats age-related neuronal dysfunction or loss (e.g., including but not limited to, mild cognitive impairment (MCI), sarcopenia, and sensory dysfunction or motor dysfunction).

In some embodiments, the present invention provides a composition comprising an inhibitor of Bak. In some embodiments, the inhibitor of Bak is an inhibitor of Bak gene expression. In some embodiments, the inhibitor of Bak is an inhibitor of Bak protein activity. In some embodiments, the inhibitor of Bak is configured to treat presbycusis. In some embodiments, the inhibitor of Bak is configured to prevent presbycusis. In some embodiments, the inhibitor of Bak is configured to treat one or more neurodegenerative disorders.

### BRIEF DESCRIPTION OF THE DRAWINGS

The foregoing summary and detailed description is better understood when read in conjunction with the accompanying drawings which are included by way of example and not by way of limitation.

FIG. 1 shows an assessment of hearing function and histological analysis of cochlea tissue. (A) Auditory Brainstem Response (ABR) threshold was measured from WT and Bak KO, and WT and Bax KO mice at 5 and 15 months of age (N=10).

*Significantly different from 5-month-old WT mice (P<0.05). **Significantly different from 15-month-old WT mice (P<0.05), error bars represent SEM. Basal cochlear turns from 5-month-old WT (B, C, D, and E), 5-month-old Bak KO (F, G, H, and I), 15-month-old WT (J, K, L, and M), and 15-month-old Bak KO (N, O, P, and Q) animals. Arrows in the lower magnification photos in the left panels indicate the SG neuronal cell, hair cell, and stria vascularis (SV) regions which are shown at higher magnification in the middle left, middle right, and right panels, respectively. Scale bar=100 μm (left panel), 20 μm (middle left, middle right, and right panel).

FIG. 2 shows the quantification of SG cell and hair cell loss. (A) SG cell densities of apical, middle, and basal cochlear turns were measured from WT and Bak KO mice at 5 and 15 months of age (N=5). (B) Outer hair cell survival rates of apical, middle, and basal cochlear turns were measured from WT and Bak KO mice at 5 and 15 months of age (N=5). (C) Inner hair cell survival rates of apical, middle, and basal cochlear turns were measured from WT and Bak KO mice at 5 and 15 months of age (N=5). *Significantly different from 5-month-old WT mice (P<0.05). **Significantly different from 15-month-old WT mice (P<0.05), error bars represent SEM.

FIG. 3 shows mRNA levels of Bak and Bax and apoptosis detection by TUNEL, cleaved Caspase-3, and Bak staining. (A) Relative cochlear mRNA expression of Bak and Bax was measured from WT and Bak KO mice at 5 and 15 months of age (N=5). (B) TUNEL-positive, cleaved Caspase-3-positive, Bak-positive cells were counted in the cochlea of WT and Bak KO mice at 5 and 15 months of age (N=5). *Significantly different from 5-month-old WT mice (P<0.05), **Significantly different from 15-month-old WT mice (P<0.05), error bars represent SEM. Cochlea from 5-month-old WT (B and C) and 15-month-old WT (D and E) mice. Arrows in left panels indicate stria vascularis regions shown at higher magnification in right panels. Arrows in lower right panel (E) indicate Bak-positive cells. Scale bar=100 μm (left panel), 20 μm (right panel).

FIG. 4 shows the mRNA sequence of human Bak (SEQ ID NO:1).

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject, unless indicated otherwise.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil-1,5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., autoimmune and chronic inflammatory disease). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, and industrial samples. Such examples are not however to be construed as limiting the sample types applicable to the present invention.

As used herein, the term “effective amount” refers to the amount of a composition (e.g., inhibitor of Bak) sufficient to effect beneficial or desired results. An effective amount can be administered in one or more administrations, applications or dosages and is not intended to be limited to a particular formulation or administration route.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administer” refer to the administration of at least two agent(s) (e.g., Bak siRNAs or antibodies and one or more other agents) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “pharmaceutical composition” refers to the combination of an active agent (e.g., Bak siRNAs and/or antibodies) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

### DETAILED DESCRIPTION OF EMBODIMENTS

In some embodiments, the present invention provides methods for prevention and treatment of presbycusis. Presbycusis, or age-related hearing loss, is the cumulative effect of aging on hearing. Presbycusis may also be known as presbycusis. It is defined as a progressive bilateral symmetrical age-related sensorineural hearing loss. Commonly, presbycusis hearing loss is most marked at higher frequencies. Although distinct from noise-induced hearing loss (NIHL), presbycusis can be exacerbated by exposure to environmental noise. Factors which may play roles in causing presbycusis include, but are not limited to heredity (e.g. early aging of the cochlea and susceptibility of the cochlea for drug insults), atherosclerosis (e.g. diminished vascularity of the cochlea), dietary habits (e.g. increased intake of saturated fat), diabetes, noise trauma, smoking, hypertension (e.g. potent vascular changes, reduction in blood supply to the cochlea, etc.), and ototoxic drugs (e.g. aspirin).

In some embodiments, the present invention provides methods for prevention and treatment of presbycusis. For example, by administering compounds that inhibit Bak activity to a patient, one can prevent and treat presbycusis. Likewise, using RNA interference technology, by administering a small interfering RNA (siRNA) that inhibit the expression of Bak gene to a patient, one can prevent and treat presbycusis. Moreover, using Gene Therapy technology, by introducing a gene that inhibits the expression of Bak gene to the cochlear cells of a patient, one can prevent and treat presbycusis. Presbycusis is associated with neural degeneration in the cochlea.

### I. Inhibition of BAK

Homo sapiens BCL2-antagonist/killer 1 (BAK1 or BAK) is described by Genbank accession number NM_{—} 001188 (FIG. 4; SEQ ID NO:1) (See e.g., the Internet Web Site of NCBI at the NIH Web Site). Bak localizes to mitochondria, and functions to induce apoptosis. It interacts with and accelerates the opening of the mitochondrial voltage-dependent anion channel, which leads to a loss in membrane potential and the release of cytochrome c. Bak also interacts with the tumor suppressor P53 after exposure to cell stress.

The present invention provides in vitro and in vivo methods of selecting inhibitors of Bak for therapeutic, clinical, or research uses. In some embodiments, candidate inhibitors (e.g., small molecules, antibodies, RNAi molecules, peptides, etc.) are administered in vitro (including in culture) or in vivo (e.g., in an animal model). The effect of the candidate compound may be assessed in any desired manner, including, but not limited to assessing enzyme activity in an in vitro activity assay or observing molecular or phenotypic changes in a cell, tissue, or animal (e.g., measuring Bak levels or assessing hearing).

Exemplary compositions useful in regulating Bak expression or activity include, but are not limited to, those described in U.S. Pat. Publ. No. 20080234201, 20080097081, 20070099215, and 20030124660 and Jurak et al., J. Virol. 82:4812 (2008), each of which is herein incorporated by reference in its entirety and those described in the below sections.

A. RNA Interference and Antisense Therapies

In some embodiments, the present invention targets the expression of Bak genes using nucleic acid based therapies. For example, in some embodiments, the present invention employs compositions comprising oligomeric antisense or RNAi compounds, particularly oligonucleotides (e.g., those described herein), for use in modulating the function of nucleic acid molecules encoding Bak genes, ultimately modulating the amount of Bak gene expressed.

1. RNA Interference (RNAi)

In some embodiments, RNAi is utilized to inhibit Bak gene function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Corners, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A 1, WO03070966A2, J Mol. Biol. 2005 May 13; 348(4):883-93, J Mol. Biol. 2005 May 13; 348(4):871-81, and Nucleic Acids Res. 2003 Aug. 1; 31 (15):4417-24, each of which is herein incorporated by reference in its entirety. In addition, software (e.g., the MWG online siMAX siRNA design tool) is commercially or publicly available for use in the selection of siRNAs. In some embodiments, commercial services (e.g., those provided by Invitrogen, Carlsbad, Calif.) are utilized in the design of siRNA sequences.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

In other embodiments, shRNA techniques (See e.g., 20080025958, herein incorporated by reference in its entirety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

2. Antisense

In other embodiments, Bak expression is modulated using antisense compounds that specifically hybridize with one or more nucleic acids encoding Bak protein. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of Bak genes. In the context of the present invention, “modulation” means either an increase (stimulation) or a decrease (inhibition) in the expression of a gene.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a Bak gene. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂—[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2. ° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Genetic Therapy

The present invention contemplates the use of any genetic manipulation for use in modulating the expression of Bak genes. Examples of genetic manipulation include, but are not limited to, gene knockout (e.g., removing the Bak gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter.

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subjects in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target proteins expressed by Bak genes. Any suitable antibody (e.g., monoclonal, polyclonal, or synthetic) may be utilized in the therapeutic methods disclosed herein. In preferred embodiments, the antibodies are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a Bak gene, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments include agents such as a coagulant, a cytokine, growth factor, bacterial endotoxin or the lipid A moiety of bacterial endotoxin. For example, in some embodiments, therapeutic agents include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted cells (e.g., Bak expressing cells) as required using known conjugation technology (See, e.g., Ghose et al., Methods Enzymol., 93:280 [1983]).

For example, in some embodiments the present invention provides immunotoxins targeting Bak genes. Immunotoxins are conjugates of a specific targeting agent typically an antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

### II. Therapeutic Applications

In some embodiments, the present invention provides therapies for presbycusis or other disorder associated with Bak gene expression. In some embodiments, therapies directly or indirectly target the Bak gene or protein of the present invention. In some embodiments, therapies indirectly target Bak expression or activity by targeting biomolecules that are functionally upstream or downstream of Bak activity or expression.

Exemplary nucleic acid, genetic and protein targeting therapies are described above or are identified in the drug screening methods described below.

The present invention is not limited to the treatment of presbycusis. In some embodiments, the present invention provides compositions and methods for prevention and treatment of neurodegenerative diseases or the effects of neurodegenerative diseases, for example those selected from the list of Alcoholism, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoffs disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, and Tabes dorsalis. In some embodiments, the present invention provides compositions and methods to prevent and treat age-associated neurodegenerative diseases (e.g. presbycusis, Alzheimer's disease, Parkinson's disease, etc.). In some embodiments, the present invention provides compositions and methods to treat age-related conditions associated with impaired neuronal activity or neuronal loss, such as mild cognitive impairment (MCI), sarcopenia, age-related sensory loss, and age-related motor dysfunction.

In some embodiments, a pharmaceutical compound with Bak inhibiting activity provides an effective agent for preventing or treating presbycusis and in some embodiments other neurodegenerative disorders, e.g., presbycusis, Alzheimer's disease, Parkinson's disease, etc. When used for the above purposes, said pharmaceutical compound may be administered via any desired oral, parenateral, topical, intervenous, transmucosal, and/or inhalation routes. The pharmaceutical compound may be administered in the form of a composition which is formulated with a pharmaceutically acceptable carrier and optional excipients, flavors, adjuvants, etc. in accordance with good pharmaceutical practice.

The composition may be in the form of a solid, semi-solid or liquid dosage form: such as tablet, capsule, pill, powder, suppository, solution, elixir, syrup, suspension, cream, lozenge, paste and spray. As those skilled in the art would recognize, depending on the chosen route of administration, the composition form of said Bak inhibitor is determined. In general, it is preferred to use a unit dosage form of the inventive inhibitor in order to achieve an easy and accurate administration of the active pharmaceutical compound. In general, the therapeutically effective pharmaceutical compound is present in such a dosage form at a concentration level ranging from about 0.5% to about 99% by weight of the total composition: i.e., in an amount sufficient to provide the desired unit dose.

A pharmaceutical compound may be administered in single or multiple doses. The particular route of administration and the dosage regimen will be determined by one of skill in keeping with the condition of the individual to be treated and said individual's response to the treatment.

For oral administration, tablets containing various excipients such as sodium citrate, calcium carbonate and dicalcium phosphate may be employed along with various disintegrants such as starch and preferably potato or tapioca starch, alginic acid and certain complex silicates, together with binding agents such as polyvinylpyrrolidone, sucrose, gelatin and acacia. Additionally, lubricating agents such as magnesium sterate, sodium lauryl sulfate and talc are often used for tabletting. Solid compositions of a similar type may also be employed as fillers in soft and hard-filled gelatin capsules; preferred materials in this connection include lactose or milk sugar as well as high molecular weight polyethylene glycols. When aqueous suspensions and/or elixirs are desired for oral administration, the active ingredient may be combined with various sweetening or flavoring agents, colorants or dyes, and, if so desired, emulsifying and/or suspending agents as well, together with such diluents as water, ethanol, propylene glycol, glycerin and various like combinations thereof.

The present invention also provides a pharmaceutical composition in a unit dosage form (e.g. for the inhibition Bak or prevention/treatment of presbycusis), comprising a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions. Suppositories for rectal administration of the pharmaceutical compound can be prepared by mixing the drug with a suitable nonirritating excipient such as cocoa butter and polyethylene glycols, which are solid at ordinary temperatures but liquid at body temperature and which therefore melt in the rectum and release the drug. Additionally, it is also possible to administer the aforesaid pharmaceutical compounds topically and this may be preferably done by way of cream, salve, jelly, paste, ointment and the like, in accordance with the standard pharmaceutical practice.

In some embodiments of the present invention, the subject is selected from the list of mammal, mouse, rat, human, etc. The subject may be stricken with presbycusis, in which case the subject may be treated in a manner not limited to those described above, to cure or alleviate presbycusis. The subject may not be stricken with presbycusis, in which case the subject may be treated in a manner not limited to those described above, to prevent, delay, or lessen the onset of presbycusis. In some embodiments, a subject of the present invention is at risk for presbycusis. In some embodiments, the subject is a patient under the care of a clinician, practitioner, or physician. In some embodiments, the patient is being treated for presbycusis or other neurodegenerative disorders.

### III. Drug Screening

In some embodiments, the present invention provides drug screening assays (e.g., to screen for compounds to treat or prevent presbycusis, or to screen for compounds which inhibit Bak). The screening methods of the present invention utilize markers identified using the methods of the present invention. For example, in some embodiments, the present invention provides methods of screening for compounds that alter (e.g., decrease or increase) the expression of the Bak gene. Compounds or agents may interfere with transcription, by interacting, for example, with the promoter region. Compounds or agents may interfere with mRNA produced from the Bak gene (e.g., by RNA interference, antisense technologies, etc.). Compounds or agents may interfere with pathways that are upstream or downstream of the biological activity of the Bak gene. In some embodiments, candidate compounds are antisense or interfering RNA agents (e.g., oligonucleotides). In other embodiments, candidate compounds are antibodies or small molecules that specifically bind to a Bak gene regulator or expression products of the present invention and inhibit its biological function.

In one screening method, candidate compounds are evaluated for their ability to alter the Bak gene expression by contacting a compound with a cell expressing a Bak gene marker and then assaying for the effect of the candidate compounds on expression. In some embodiments, the effect of candidate compounds on is assayed for by detecting the level of Bak gene mRNA expressed by the cell. mRNA expression can be detected by any suitable method.

In other embodiments, the effect of candidate compounds on expression of Bak gene is assayed by measuring the level of polypeptide. The level of polypeptide expressed can be measured using any suitable method, including but not limited to, those disclosed herein.

In some embodiments, the present invention provides screening methods for identifying modulators, i.e., candidate or test compounds or agents (e.g., proteins, peptides, peptidomimetics, peptoids, small molecules or other drugs) which bind to the Bak gene, have an inhibitory effect on the Bak gene, or have an inhibitory effect on Bak expression or activity. Compounds thus identified can be used to modulate the activity of Bak either directly or indirectly in a therapeutic protocol. Compounds that inhibit the activity or expression of Bak are useful in the treatment of presbycusis and/or neurodegenerative disorders.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection.

In one embodiment, an assay is a cell-based assay in which a cell that expresses a Bak mRNA, or protein, or biologically active portion thereof is contacted with a test compound, and the ability of the test compound to the modulate Bak activity is determined. In another embodiment, a cell-free assay is provided in which a Bak protein or biologically active portion thereof is contacted with a test compound and the ability of the test compound to bind Bak protein, mRNA, or biologically active portion thereof is evaluated.

This invention further pertains to novel agents identified by the above-described screening assays (See e.g., below description of therapies). Accordingly, it is within the scope of this invention to further use an agent identified as described herein (e.g., a Bak inhibitory agent, an antisense nucleic acid molecule, a siRNA molecule, a Bak specific antibody, etc.) in an appropriate animal model to determine the efficacy, toxicity, side effects, or mechanism of action, of treatment with such an agent. Furthermore, novel agents identified by the above-described screening assays can be, e.g., used for treatments as described herein.

### EXPERIMENTAL

### Example 1

### Compositions and Methods for Preventing and Treating Presbycusis

Animals. Male and female Bak KO (B6.129-Bak1tm1Thsn/J), Bax KO (B6.129×1-Baxtm1Sjk/J), and C57BL/6J (B6) mice were used (Jackson Laboratory, Bar Harbor, Me.). The B6 mice were used as wild-type (WT) controls for Bak KO and Bax KO mice as these KO mice were backcrossed for 5-21 generations onto the C57BL/6 background. Mice were housed in the AAALAC-approved Animal Facility in the Genetics and Biotechnology Center of the University of Wisconsin-Madison. Experiments were performed in accordance with protocols approved by the University of Wisconsin-Madison Institutional Animal Care and Use Committee.

Assessment of hearing function. Detailed protocols for auditory brainstem response (ABR) measurements were performed as previously described (Yamasoba et all., Neurosci Lett. 395, 18-22 (2006), herein incorporated by reference in its entirety). ABRs were measured with a tone burst stimulus (8, 16, and 32 kHz) using an ABR recording system (Intelligent Hearing System, Miami, Fla.). Mice were anesthetized with a mixture of xylazine hydrochloride (10 mg/kg, i.m.) and ketamine hydrochloride (40 mg/kg, i.m.) (10 male mice per group (N=10)). Following the Bak KO, Bax KO, and WT mouse hearing measurements, the same mice were used to conduct histological, TUNEL, immunohistochemical, and quantitative RT-PCR analyses. All data were reported as mean±S.E.M.

Histological analysis. Detailed protocols for histological assessment have been described (Yamasoba et al., Neurosci Lett. 395, 18-22 (2006), herein incorporated by reference in its entirety). The temporal bone was excised from the head and divided into cochlear and vestibular parts. The cochlea was then excised, immersed in a fixative containing 4% paraformaldehyde in phosphate buffer for 1 day, and decalcified in 10% ethylenediaminetetraacetic acid for 1 week. The paraffin-embedded specimens were sliced into 4 μm sections, mounted on silane-coated slides, stained with Haematoxylin and Eosin (HE), and observed under a light microscope (Leica Microsystems, Bannockburn, Ill.). The Rosenthal's canal was divided into three regions: apical, middle and basal (Keithley et al., Hear Res. 188, 21-28 (2004), herein incorporated by reference in its entirety) and the three turns were used for evaluation of cochlear histology (five male mice per group (N=5)). Five modiolar sections were obtained in every fifth section from one unilateral cochlea were evaluated per mouse. The same animals were used for spiral ganglion (SG) cell counting, hair cell counting, TUNEL staining, Caspase-3 immunostaining, and Bak immunostaining.

Measurement of spiral ganglion cell density. SG cells were counted in the apical, middle, and basal turn of the cochlear sections using a 20× objective (Keithley et al., Hear Res. 188, 21-28 (2004).). Cells were identified by the presence of a nucleus. The corresponding area of Rosenthal's canal was measured on digital photomicrographs of each canal profile. The perimeter of the canal was traced with a cursor using ImageJ software (Rasband, W. S., ImageJ, U.S. National Institutes of Health, Bethesda, Md., USA, http://rsb.info.nih.gov/ij/, 1997-2007.). The area was calculated within the outline. The SGC density was calculated as the number of SGCs per mm². Five sections of the unilateral apical, middle, and basal turns were evaluated in one cochlea per mouse (5 male mice per group (N=5)). All data were reported as mean±S.E.M.

Measurement of hair cell survival rate. Outer hair cells (OHCs) and inner hair cells (IHCs) were counted in the apical, middle, and basal turns of the cochlear sections using a 40× objective. Cells were identified by the presence of a nucleus. The outer hair cell survival rate (%) was calculated as (the number of OHCs per 3)×100. The inner hair cell survival rate (%) was calculated as (the number of IHCs per 1)×100. Five sections of the unilateral apical, middle, and basal turns were evaluated in one cochlea per mouse (five male mice per group (N=5)). All data were reported as mean±S.E.M.

Quantitative RT-PCR. Detailed protocols for quantitative RT-PCR (QRT-PCR) analysis have been described (Someya et al., Neurobiol Aging. 29, 1080-1092 (2008), herein incorporated by reference in its entirety). The cochleae were dissected, frozen in liquid N2, and stored at −80° C. Total RNA was extracted from the frozen cochlear tissues using the TRIZOL reagent (Life Technologies, Grand Island, N.Y.) as previously described (Lee et al., Science. 285, 1390-1393 (1999), herein incorporated by reference in its entirety). Detection of mRNA was carried out with the TaqMan EZ RT-PCR kit using an Applied Biosystems Prism 7000 Sequence Detection System (Applied Biosystems, Foster City, Calif.). β-Actin was used as an internal standard. Oligonucleotide primers and MGB fluorescent probes (TaqMan Gene Expression Assays) were purchased from Applied Biosystems. Each sample used for QRT-PCR analysis consisted of the two cochleae pooled from one mouse (five male mice per group (N=5)). All data were reported as mean±SEM.

TUNEL staining. TUNEL staining for apoptotic nuclei was performed using a DeadEnd Colormetric TUNEL System (Promega, Madison, Wis.) according to the manufacturer's instructions. Labeling reactions were performed for 60 min at 25° C. in a humidified chamber. Color development was accomplished with diaminobenzidine for 8 min. Duplicate sections were counter-stained with hematoxylin and a coverglass was mounted to each slide with Permount.

TUNEL-positive cells were counted in the apical, middle, and basal turns of the cochlear sections using a 20× objective. Cells were identified by the presence of a brown-stained nucleus in the SG regions. The corresponding area of Rosenthal's canal was measured on digital photomicrographs of each canal profile in the same manner as described in the SG cell counting section. The TUNEL-positive (the density of TUNEL-positive cells) was calculated as the number of TUNEL-positive cells per mm². Five sections of the unilateral apical, middle, and basal turns were evaluated in one cochlea per mouse (five male mice per group (N=5)). All data were reported as mean±S.E.M.

Cleaved caspase-3 staining. Detailed protocols for immunohistochemical staining have been described (Sakamoto et al., Laryngoscope. 114, 1988-91 (2004), herein incorporated by reference in its entirety). Immunohistochemical staining for caspase-3 was performed using the rabbit polyclonal cleaved caspase-3 antibody (Cell Signaling, Danvers, Mass.) according to the manufacturer's instructions. Briefly, fixed sections were deparafinized, incubated in 3% H2O2 in PBS (phosphate-buffered saline) for 10 min, and incubated in blocking buffer (Pierce, Rockford, Ill.) for 60 min. The sections were then incubated in a moist chamber for 60 min at 25° C. with primary rabbit polyclonal cleaved-caspase-3 antibody diluted 1:400 in the blocking buffer. Negative controls were established by replacing primary antibody with the blocking buffer. The sections were then incubated with biotinylated rabbit secondary antibody diluted in 1:400 in PBS (Vector, Burlingame, Calif.) for 60 min. The sections were treated with ABC solution (Vector) for 60 min and incubated with DAB substrate (Vector) for 3 min. Counterstaining was carried out with hematoxylin.

Cleaved caspase-3-positive cells were counted in the apical, middle, and basal turns of the cochlear sections using a 20× objective. Cells were identified by the presence of a brown-stained cell in the SG regions. Cleaved caspase-3-positive cell counting and area calculation were performed in the same manner as described in the TUNEL staining section. The density of cleaved caspase-3-positive cells was calculated as the number of cleaved caspase-3-positive cells per mm². Five sections of the unilateral apical, middle, and basal turns were evaluated in one cochlea per mouse (five male mice per group (N=5)). All data were reported as mean±S.E.M.

Bak staining. Immunohistochemical staining for Bak was performed in the same manner as described in the cleaved caspase-3 staining section. Fixed sections were incubated in a moist chamber for 60 min at 25° C. with primary rabbit monoclonal Bak antibody (Abcam, Cambridge, Mass.) diluted 1:100 in the blocking buffer. Bak-positive cells were counted in the apical, middle, and basal turns of the cochlear sections using a 20× objective. Cells were identified by the presence of a brown-stained cell in the stria vascularis. Bak-positive cell counting and area calculation were performed in the same manner as described in the TUNEL staining section. The density of Bak-positive cells was calculated as the number of Bak-positive cells per mm². Five sections of the unilateral apical, middle, and basal turns were evaluated in one cochlea per mouse (five male mice per group (N=5)). All data were reported as mean±S.E.M.

### Example 2

### Preventing and Treating Prebycusis

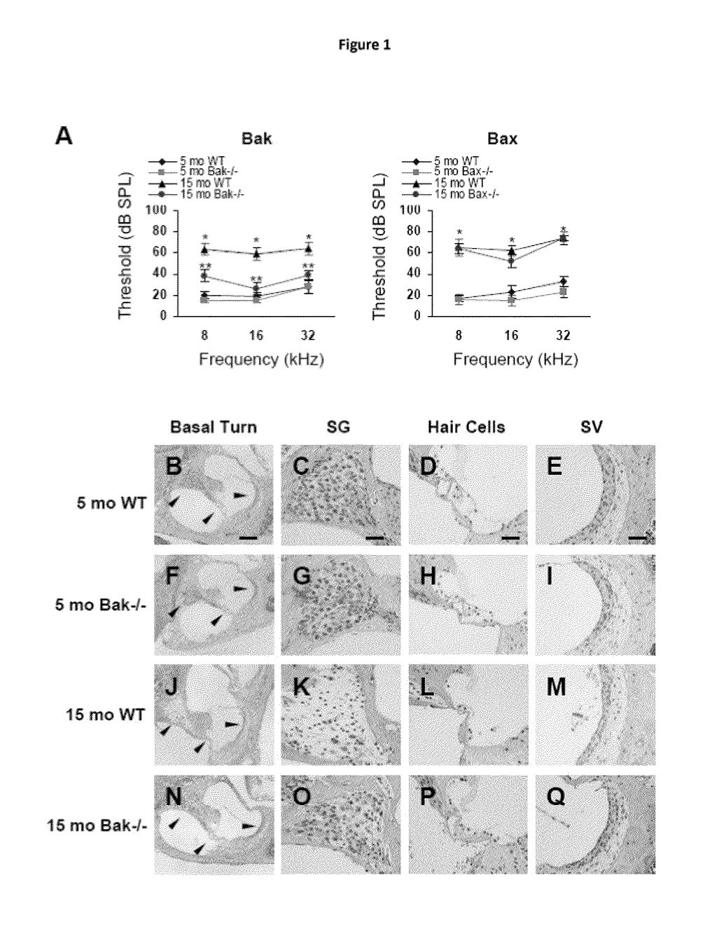

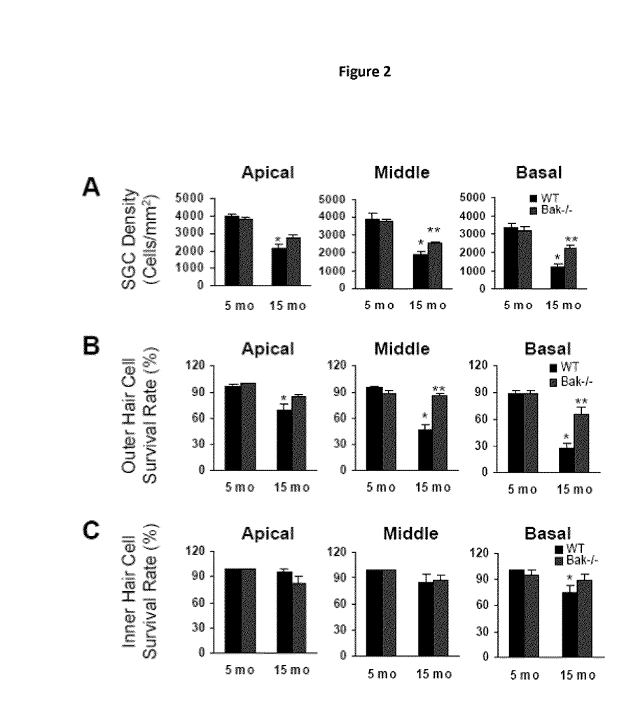

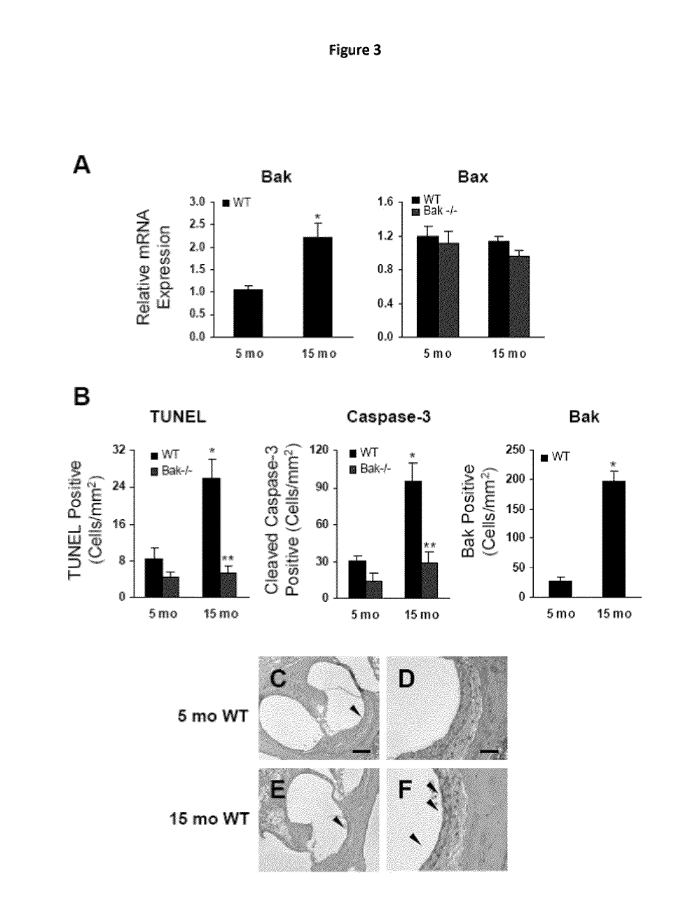

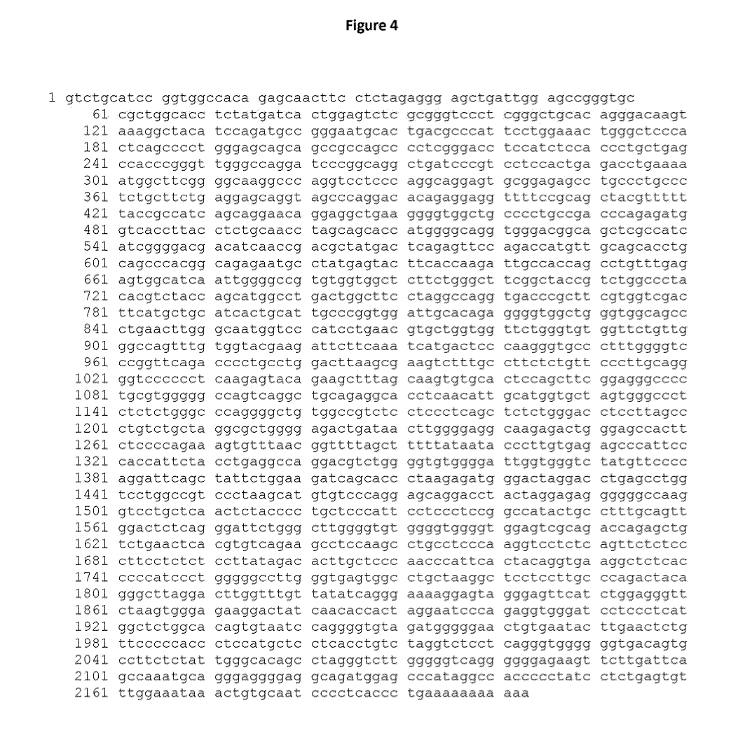

In experiments conducted during development of embodiments of the present invention, auditory brainstem response (ABR) hearing tests were conducted in 5-month-old and 15-month-old Bak KO, Bax KO, and WT mice, all in the same B6 background to examine whether proapoptotic Bak and/or Bax play a causal role in the pathogenesis of AHL. The middle-age Bak KO mice retained normal hearing at the middle frequency (16 kHz) and displayed slight to mild hearing loss at the low (8 kHz) and high (32 kHz) frequencies, while age-matched WT mice displayed severe hearing loss at all the frequencies measured (SEE FIG. 1A). In contrast, both middle-age Bax KO and WT mice displayed severe hearing loss at all the frequencies (SEE FIG. 1A). Histological analysis was conducted on the cochleae from 5-month-old and 15-month-old Bak KO, Bax KO, and WT mice to examine tissue damage responsible for the hearing function loss. In agreement with the ABR results, middle-age Bak KO cochleae displayed only minor loss of SG and hair cells, and no or slight difference in thickness of stria vascularis (SEE FIG. 1N-Q), while WT cochleae displayed severe loss of SG and hair cells, and significant reduction in thickness of stria vascularis by 15 months of age (SEE FIG. 1B-E and J-M). Middle-age Bax KO cochleae displayed severe cochlear degeneration. Decreased SG cell density and hair cell number are hallmarks of presbycusis (Yamasoba et al., Hear Res 226, 185 (2007).). The mean SG cell densities of middle and basal cochlear turns from middle-age Bak KO mice were significantly higher than those of age-matched WT mice (SEE FIG. 2A). Moreover, the mean outer hair cell survival rates of middle and basal cochlear turns from middle-age Bak KO mice were significantly higher than those of age-matched WT mice (SEE FIG. 2B), while no significant change was observed in inner hair cell survival rates between middle-age WT and Bak KO (FIG. 2C). Thus, experiments performed during the development of embodiments of the present invention determined that Bak, but not Bax, is required for the pathogenesis of AHL, although the present invention is not limited to any particular mechanism of action and an understanding of the mechanism of action is not necessary to practice the present invention.

Experiments were conducted during the development of embodiments of the invention to measure relative mRNA levels of Bak and Bax genes in the cochleae of 5-month-old and 15-month-old Bak KO and WT mice by quantitative RT-PCR to examine whether levels of Bcl-2 members are increased during aging in cochlea. The mean relative mRNA expression of Bak was found to increase during aging in the WT cochlea (SEE FIG. 3A). No significant changes were observed in relative mRNA levels of Bax between young and middle-age WT cochlea (SEE FIG. 3A). Immunohistochemical measurements of Bak protein levels in the WT cochlea were performed and found that levels of Bak were increased during aging in the WT cochlea (SEE FIG. 3B). Thus, aging leads to increased levels of Bak, but not Bax, in cochlea, although the present invention is not limited to any particular mechanism of action and an understanding of the mechanism of action is not necessary to practice the present invention.

In the mitochondrial pathway of apoptosis, Bak is postulated to play a major role in promoting apoptosis, leading to activation of caspase-3 (Youle & Strasser, Nat Rev Mol Cell Biol 9, 47 (2008), Lindsten et al., Mol Cell 6, 1389 (2000).), although the present invention is not limited to any particular mechanism of action and an understanding of the mechanism of action is not necessary to practice the present invention. TUNEL staining was conducted to measure levels of nuclear DNA fragmentation, a key feature of apoptosis to examine whether Bak plays an essential role in promoting apoptosis in aging cochlea (Kujoth et al., Science 309, 481 (2005), Youle & Strasser, Nat Rev Mol Cell Biol 9, 47 (2008).), in 5-month-old and 15-month-old Bak KO and WT mice. Consistent with the findings of increased proapoptotic Bak, aging resulted in increased levels of TUNEL-positive cells in the WT cochlea (SEE FIG. 3B). No significant difference was observed in levels of TUNEL-positive cells between young WT and middle-age Bak KO cochlea. Apoptosis was further investigated by immunohistochemical staining for active caspase-3. Caspase-3 is one of the key executioner caspases, although the present invention is not limited to any particular mechanism of action and an understanding of the mechanism of action is not necessary to practice the present invention (Youle & Strasser, Nat Rev Mol Cell Biol 9, 47 (2008).). Aging resulted in increased levels of active caspase-3-positive cells in the WT cochlea (FIG. 3B), but there was no significant difference between young WT and middle-age Bak KO cochlea. Moreover, the localization of Bak and the levels of Bak-positive cells in aged cochlea were investigated by immunohistochemical staining, and levels of Bak-positive cells were increased during aging in the WT cochlea (SEE FIG. 3B). Bak localized primarily in the stria vascularis region of the middle-age WT cochlea (SEE FIG. 3B-F), consistent with findings that age-related degeneration of stria vascularis is the most prominent anatomical characteristic of presbycusis in humans (Gates & Mills, Lancet 366, 1111 (2005).). In agreement with the apoptosis test results, the histological results clearly demonstrated that aging leads to decreased numbers of cells in the middle-age WT cochlea, but not in the age-matched Bak KO cochlea (SEE FI. 1J-Q).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of providing medical treatment to a subject having presbycusis comprising inhibiting Bak in said subject.

2. The method of claim 1, wherein said providing medical treatment comprises preventing presbycusis.

3. The method of claim 1, wherein said providing medical treatment comprises treating presbycusis.

4. The method of claim 1, wherein said inhibiting Bak comprises inhibiting expression of the Bak gene.

5. The method of claim 1, wherein said inhibiting Bak comprises inhibiting Bak protein activity.

6. The method of claim 1, wherein said inhibiting Bak comprises administering Bak-inhibiting compounds to said subject.

7. The method of claim 1, wherein said inhibiting Bak comprises administering siRNA to said subject.

8. The method of claim 1, wherein said inhibiting Bak comprises introducing a gene to said subject.

9. The method of claim 8, wherein said gene inhibits the expression of said Bak gene in the cochlear cells of said subject.

10. The method of claim 1, wherein said inhibiting Bak in said subject treats one or more neurodegenerative diseases.

11. The method of claim 1, wherein said inhibiting Bak in said subject prevents one or more neurodegenerative diseases.

12. The method of claim 1, wherein said subject is a mouse.

13. The method of claim 1, wherein said subject is a human.

14. The method of claim 1, wherein said inhibiting Bak in said subject comprises inhibiting Bak in cochlear cells.

15. The method of claim 1, wherein said subject is a companion animal.

16. The method of claim 15, wherein said subject is selected from the group consisting of a dog and a cat.

17. The method of claim 1, wherein said inhibiting bak in said subject prevents or treats age-related neuronal dysfunction or loss.

18. The method of claim 17, wherein said age related neuronal dysfunction or loss is selected from the group consisting of mild cognitive impairment (MCI), sarcopenia, and sensory dysfunction and motor dysfunction.
